(19) United States
(12) Patent Application Publication
(10) Pub. No.: US 2012/0136406 A1
(43) Pub. Date: May 31, 2012

(54) Systems and Methods for Determining Optimal Atrioventricular Pacing Delays Based on Cardiomechanical Delays

(75) Inventor: Xiaoyi Min, Thousand Oaks, CA (US)

(73) Assignee: PACESETTER, INC., Sylmar, CA (US)

(21) Appl. No.: 12/957,142

(22) Filed: Nov. 30, 2010

(51) Int. Cl. A61N 1/365

(52) U.S. Cl. 607/25

(57) ABSTRACT

Techniques are provided for use with implantable medical devices such as pacemakers for optimizing atrioventricular (AV) pacing delays for use with cardiac resynchronization therapy (CRT). In one example, the end of atrial mechanical contraction and the onset of isovolumic ventricular mechanical contraction are detected within a patient in which the device is implanted based on cardiomechanical signals, such as cardiogenic impedance (Z) signals, S1 heart sounds or left atrial pressure (LAP) signals. Then, a cardiomechanical time delay (MC_AV) between the end of atrial contraction and the onset of isovolumic ventricular contraction is determined. AV pacing delays are set based on MC_AV to align the end an atrial kick with the onset of isovolumic ventricular contraction. Thereafter, pacing is controlled based on the AV pacing delays.

### FIELD OF THE INVENTION

The invention generally relates to implantable cardiac rhythm management devices such as pacemakers and implantable cardioverter-defibrillators (ICDs) and cardiac resynchronization therapy (CRT) devices and, in particular, to techniques for determining preferred or optimal atrioventricular (AV) pacing delays for use in pacing the heart using such devices.

### BACKGROUND OF THE INVENTION

Clinical studies related to cardiac pacing have shown that an optimal atrioventricular pacing delay (e.g., AV delay) and/or an optimal interventricular pacing delay (e.g., VV delay) can improve cardiac performance. (Note that the term “AV delay” as it is used herein includes atrioventricular pacing delays following intrinsic atrial events—sometimes specifically referred to as PV delays—as well as atrioventricular pacing delays following paced atrial events. That is, unless otherwise noted, “AV delay” encompasses AV/PV delays.) However, such optimal delays depend on a variety of factors that may vary over time. Thus, what is “optimal” may vary over time. An optimization of AV/PV pacing delay and/or VV pacing delay may be performed at implantation and sometimes, a re-optimization may be performed during a follow-up consultation. While such optimizations are beneficial, the benefits may not last due to changes in various factors related to device and/or cardiac function.

The following patents and patent applications set forth various systems and methods for allowing a pacemaker, ICD, CRT or other cardiac rhythm management (CRM) device to determine and/or adjust AV/VV pacing delays so as to help maintain the pacing delays at optimal values: U.S. patent application Ser. No. 10/986,273, filed Nov. 10, 2004 (Attorney Docket No. A03P1074US02), now U.S. Pat. No. 7,590,446; and U.S. patent application Ser. No. 11/952,743, filed Dec. 7, 2007 (Attorney Docket No. A07P1179). See, also, U.S. patent application Ser. No. 12/328,605, filed Dec. 4, 2008, entitled “Systems and Methods for Controlling Ventricular Pacing in Patients with Long Intra-Atrial Conduction Delays” (Attorney Docket No. A08P1067); U.S. patent application Ser. No. 12/132,563, filed Jun. 3, 2008, entitled “Systems and Methods for determining Intra-Atrial Conduction Delays using Multi-Pole Left Ventricular Pacing/Sensing Leads” (Attorney Docket No. A08P1021), now U.S. Pub. App. 2009/0299423A1; and U.S. patent application Ser. No. 12/639,881, filed Dec. 16, 2009, entitled “Systems and Methods for Determining Ventricular Pacing Sites for use with Multi-Pole Leads” (Attorney Docket No. A09P1034US01.) See, further, U.S. Pat. No. 7,248,925, to Bruhns et al., entitled “System and Method for Determining Optimal Atrioventricular Delay based on Intrinsic Conduction Delays.” At least some of the techniques are implemented within the QuickOpt™ systems of St. Jude Medical.

In particular, intracardiac electrogram (IEGM)-based techniques are set forth within at least some of these documents for exploiting various inter-atrial and interventricular conduction delays observed within the IEGM to determine preferred or optimal AV pacing delays for use in delivering CRT. Briefly, CRT seeks to normalize asynchronous cardiac electrical activation and resultant asynchronous contractions associated with congestive heart failure (CHF) by delivering synchronized pacing stimulus to both ventricles. The stimulus is synchronized so as to improve overall cardiac function. This may have the additional beneficial effect of reducing the susceptibility to life-threatening tachyarrhythmias.

It would be desirable to provide improvements in the determination of preferred or optimal AV pacing delays for use with CRT and aspects of the present invention are directed to this general goal.

### SUMMARY OF THE INVENTION

In an exemplary embodiment, a method is provided for controlling the delivery of cardiac pacing therapy by an implantable cardiac rhythm management device for implant within a patient. Briefly, atrial mechanical contractions are detected within the patient based on cardiomechanical signals sensed by the device. The onset of isovolumic ventricular mechanical contractions are also detected within the patient based on the cardiomechanical signals. Then, a cardiomechanical time delay between the atrial contractions and the onset of isovolumic ventricular contractions is determined. Preferably, the end of the atrial mechanical contraction is used to measure the time delay. AV pacing delays are then set based on the time delay to, for example, align the end of the atrial kick provided by the contracting atria with the onset of isovolumic ventricular mechanical contraction so as to improve hemodynamics. Thereafter, pacing is controlled based on the AV pacing delays. By setting AV pacing delays based on cardiomechanical time delays that account for the timing of atrial and ventricular mechanical contractions, the AV delays can be more easily and effectively timed. Again, it is noted that the term “AV delay” as it is used herein broadly encompasses both AV and PV delays.

In an illustrative example, the implantable device is a pacemaker, ICD or CRT device. The cardiomechanical signals sensed by the device include cardiogenic impedance, left atrial pressure (LAP), right ventricular pressure (RVP), left ventricular pressure (LVP), photo-plethysmography (PPG) signals, and/or heart sounds. The cardiomechanical delay between the end of atrial contraction and the onset of isovolumic ventricular contraction is detected based on one or more of these signals. This delay is referred to herein as ‘MC_AV’.

In an embodiment where impedance is exploited, values representative of electrical cardiogenic impedance (Z) are detected along vectors within the heart of the patient. Preferably, separate vectors are used to detect the atrial contractions as opposed to ventricular contractions. In one specific example, wherein the device is equipped with at least a right atrial (RA) lead and a multi-pole left ventricular (LV) lead, the atrial impedance vector includes an electrical current injection vector between an RA tip electrode and an LV proximal ring electrode and an impedance-responsive voltage sensing vector between the RA tip electrode and an LV middle ring electrode. Herein, the signal sensed along the impedance-responsive voltage sensing vector is referred to as Z as it is representative of impedance.) In another example, wherein the device is equipped with a bipolar RA lead and a multi-pole LV lead, the atrial impedance vectors include a bipolar electrical current injection vector between RA tip and ring electrodes and a bipolar impedance-responsive voltage sensing vector between adjacent LV ring electrodes. Insofar as detecting ventricular cardiogenic impedance is concerned, in one example a bipolar electrical current injection vector is employed between RV tip and LV tip electrodes along with a bipolar impedance-responsive voltage sensing vector between LV ring and RV ring electrodes. In another ventricular cardiogenic impedance example, the bipolar electrical current injection vector is instead between RV tip and ring electrodes and the bipolar impedance-responsive voltage sensing vector is between LV tip and ring electrodes.

To detect the end of atrial mechanical contraction based on impedance, the device can identify the point where the magnitude of the measured atrial cardiogenic impedance signal falls below a predetermined threshold. The onset of isovolumic ventricular contraction can be detected along the ventricular cardiogenic impedance vector by, e.g., exploiting a time rate of change in ventricular cardiogenic impedance (i.e. dZ/dt). The onset of isovolumic ventricular mechanical contraction is deemed to correspond to the timing of the peak rate of change in dZ/dt (i.e. MAX(d²Z/dt²)) or other suitable parameters. This value is also typically close to the peak in the QRS. The onset of isovolumic ventricular mechanical contraction can also be detected based on heart sounds. For example, the peak of an S1 heart sound can be detected using an implantable acoustic sensor. The onset of isovolumic ventricular mechanical contraction is deemed to correspond to the timing of the peak of S1. In an embodiment wherein LAP is exploited, first and second peaks in LAP are detected (near the QRS complex of the LV IEGM) using an implantable pressure sensor or sensing technique. The onset of isovolumic contraction is deemed to correspond to the timing of a valley or trough between the first and second peaks. Insofar as LVP, RVP and PPG are concerned, the time rate of change in the signal can be detected, with the onset of isovolumic contraction deemed to coincide with the peak in the rate of change of the signal (i.e. d²LVP/dt², dRVP²/dt² or dPPG²/dt² or other suitable parameters)

To determine the preferred or optimal value for the AV pacing delays, initial or baseline AV delays can be determined using existing IEGM-based optimization techniques, such as one of the aforementioned QuickOpt techniques. Then, while pacing is delivered using the initial AV delays and with VV set to zero, the value for MC_AV is measured (i.e. the time delay between the end of atrial mechanical contraction and the onset of isovolumic ventricular mechanical contraction.) The optimal AV pacing delay (AV delay1) is then set equal to a baseline AV delay minus MC_AV plus an offset (which in some cases is set to zero) to align the end of a paced atrial mechanical kick with the onset of ventricular contraction. Preferably, pacing is then delivered using the optimal AV pacing delay and a new value for MC_AV is measured to confirm that the new value of MC_AV is small enough or meets a predetermined criteria (e.g. <20 ms) or is otherwise hemodynamically acceptable. This may be determined, for example, by comparing the new MC_AV delay against a threshold indicative of an acceptable MC_AV delay. In some examples, the AV delays are further refined based on the widths and strengths of the atrial and ventricular mechanical contractions, as derived from cardiomechanical signal parameters.

In embodiments where the device is equipped to detect LAP, the MC_AV time delay (i.e. the delay between the end of atrial mechanical contraction and the onset of isovolumic ventricular mechanical contraction) can be determined based on a delay between a first peak and a first valley in LAP near a QRS complex (as detected using an IEGM signal.) If the device is also equipped to detect S1 heart sounds, MC_AV can be determined based on a delay between the first peak in LAP and a first peak in the S1 heart sound near the QRS. If the device is equipped to detect LVP, RVP or PPG signals, MC_AV can be determined based on a delay between the first peak in LAP and a sharp increase in LVP, RVP or PPG (as indicated by MAX(d²LVP/dt²), MAC(dRVP²/dt²) or MAX(dPPG²/dt².))

System and method implementations of the various exemplary embodiments are presented herein.

### BRIEF DESCRIPTION OF THE DRAWINGS

Features and advantages of the described implementations can be more readily understood by reference to the following description taken in conjunction with the accompanying drawings.

FIG. 1 illustrates pertinent components of an implantable medical system having a pacemaker, ICD or CRT device equipped to optimize AV pacing delays based on certain cardiomechanical delays detected within a patient in which the device is implanted;

FIG. 2 provides an overview of a technique for setting preferred or optimal AV pacing delays that may be performed by the system of FIG. 1;

FIG. 3 illustrates an exemplary technique for use with the general technique of FIG. 2 for detecting atrial mechanical contractions using impedance;

FIG. 4 illustrates an exemplary technique for use with the general technique of FIG. 2 for detecting ventricular mechanical contractions using impedance;

FIG. 5 illustrates an exemplary technique for setting AV delays based on an MC_AV delay measured between the atrial and ventricular mechanical contractions detected using the techniques of FIGS. 3 and 4;

FIG. 6 illustrates echo Doppler waveforms, impedance signals and electrocardiogram (ECG) waveforms, and particularly illustrating the MC_AV delay detected and exploited by the techniques of FIGS. 3-5;

FIG. 7 illustrates another exemplary implementation of the general technique of FIG. 2 wherein non-impedance cardiomechanical signals are exploited, such as LAP, LVP and heart sounds;

FIG. 8 is a graph illustrating additional exemplary LAP, LVP and heart sound signals that can be exploited by the technique of FIG. 7, as well as ECG waveforms;

FIG. 9 is a simplified, partly cutaway view, illustrating the device of FIG. 1 along with at set of leads implanted into the heart of the patient;

FIG. 10 is a functional block diagram of the pacer/ICD of FIG. 9, illustrating basic circuit elements that provide cardioversion, defibrillation and/or pacing stimulation in the heart an particularly illustrating on-board optimization components for performing the various AV optimization techniques;

FIG. 11 is a functional block diagram illustrating components of the external device programmer of FIG. 1 and particularly illustrating programmer-based optimization components for controlling the various AV optimization techniques.

### DETAILED DESCRIPTION OF THE PREFERRED EMBODIMENTS

The following description includes the best mode presently contemplated for practicing the invention. This description is not to be taken in a limiting sense but is made merely to describe general principles of the invention. The scope of the invention should be ascertained with reference to the issued claims. In the description of the invention that follows, like numerals or reference designators will be used to refer to like parts or elements throughout.

### Overview of Implantable Medical System

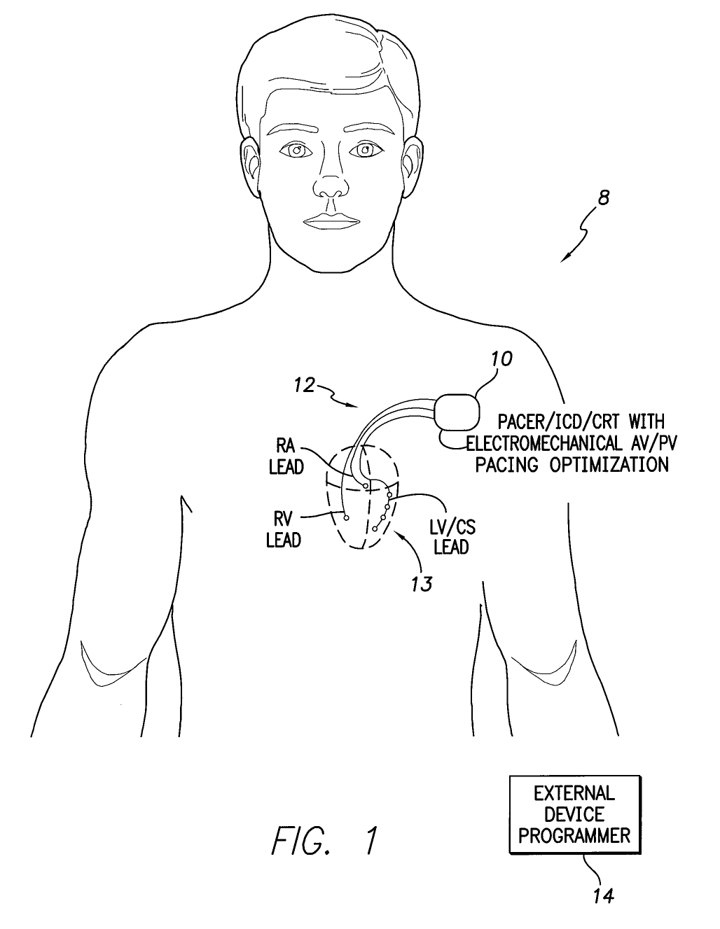

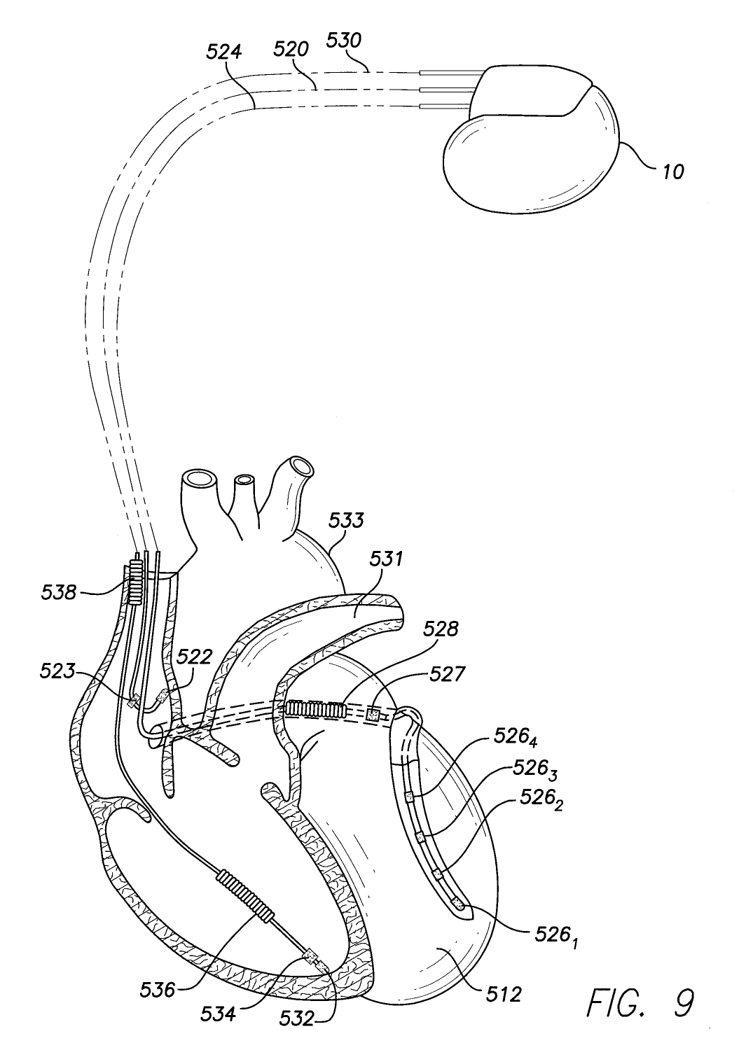

FIG. 1 illustrates an implantable cardiac rhythm management system 8 capable of performing rapid optimization of AV pacing delays, alone or in combination with an external programmer 14. The implantable medical system 8 includes a pacer/ICD/CRT device 10 or other cardiac rhythm management device equipped with one or more leads 12 implanted on or within the heart of the patient, including a multi-pole LV lead implanted via the coronary sinus (CS). In FIG. 1, a stylized representation of the set of leads is provided. To illustrate the multi-pole configuration of the LV lead, a set of electrodes 13 is shown distributed along the LV lead. The RV and RA leads are each shown with a single electrode, though each of those leads may include additional electrodes as well, such as tip/ring electrode pairs. Still further, the LV lead can also include one or more left atrial (LA) electrodes mounted on or in the LA via the CS. See FIG. 9 for a more complete and accurate illustration of various exemplary leads, including an exemplary multi-pole LV lead. It is noted that a multi-pole lead is not required, though such a lead provides advantages in terms of pacing/sensing vector selection. In the alternative, an LV lead with a ring electrode or a pair of electrodes near the LA or inside distal CS can instead be used. If a standard LV lead is employed, atrial mechanical contractions can typically be extracted from ventricular impedance vector components.

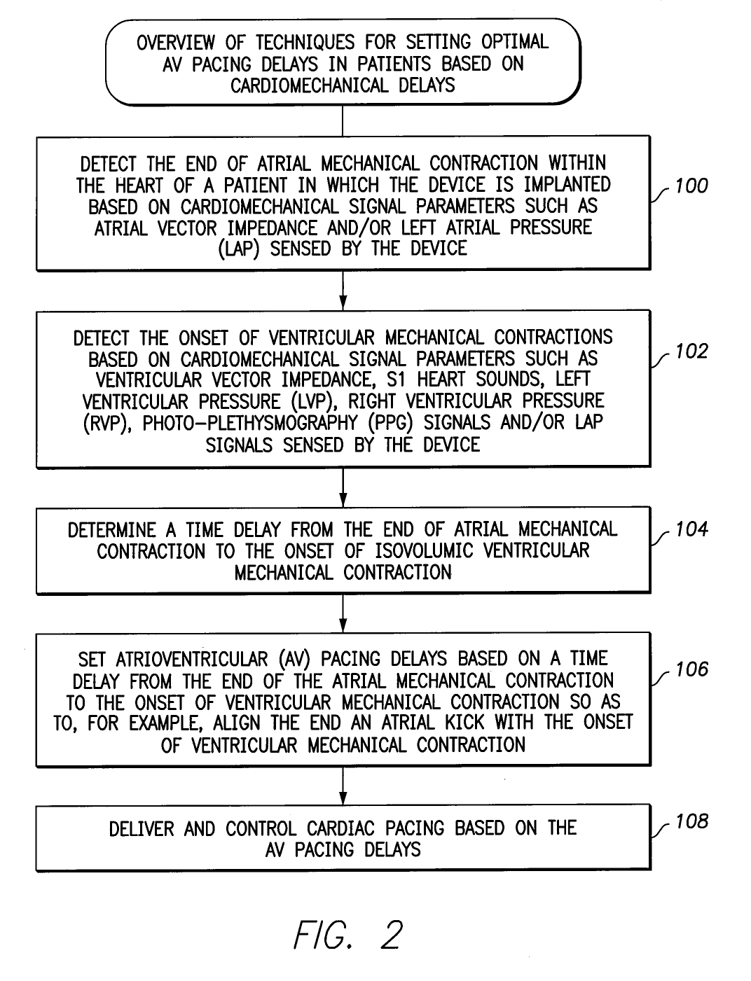

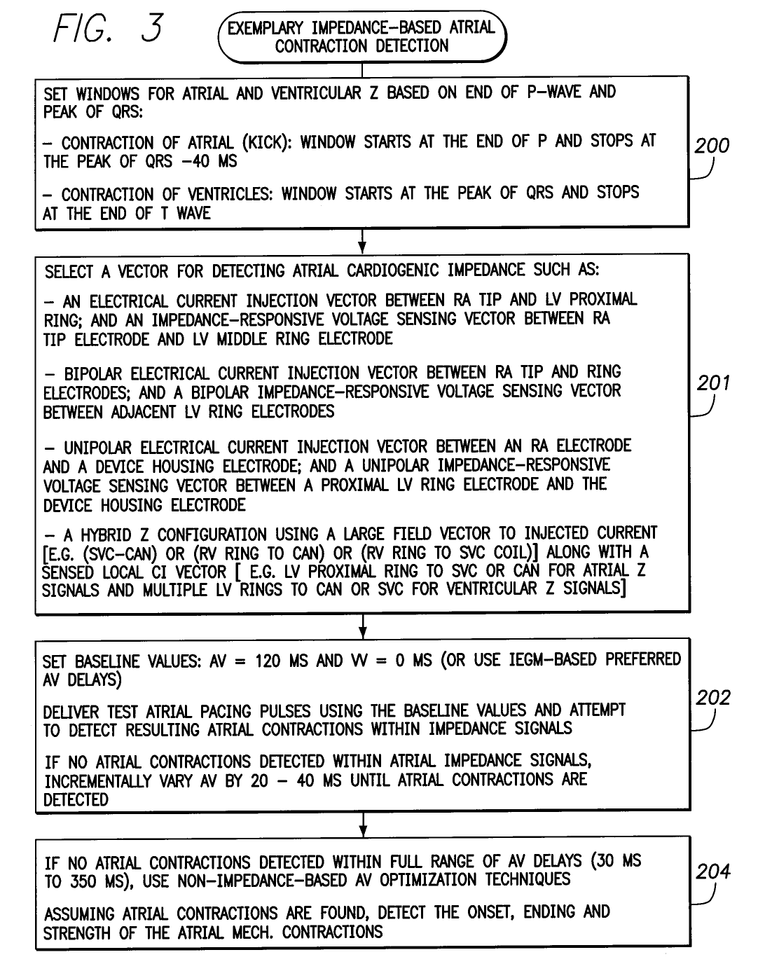

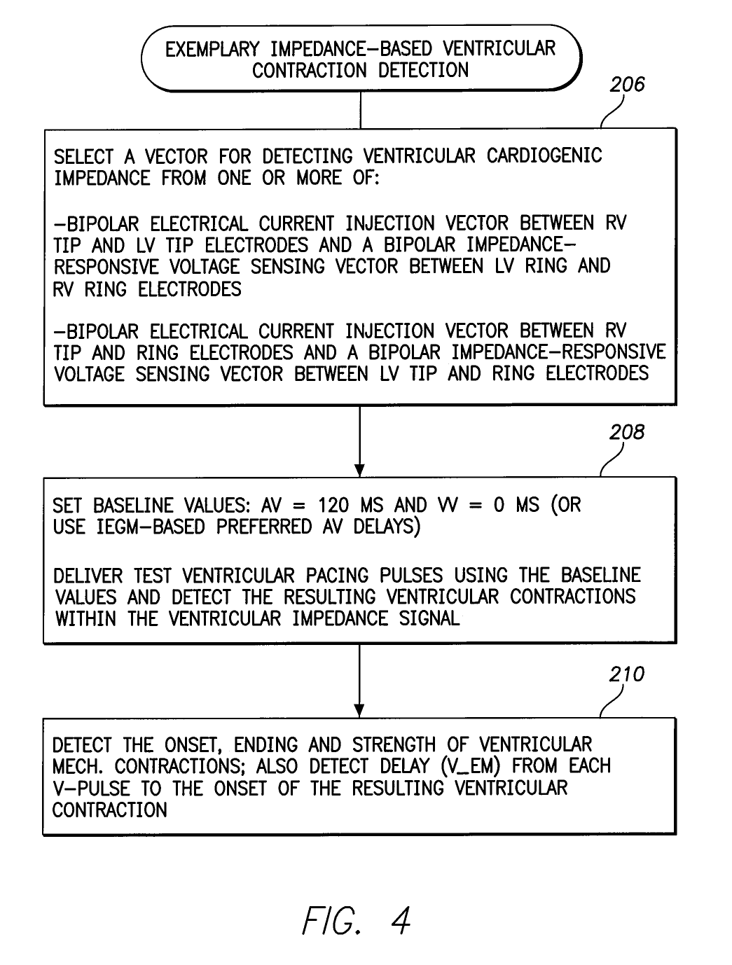

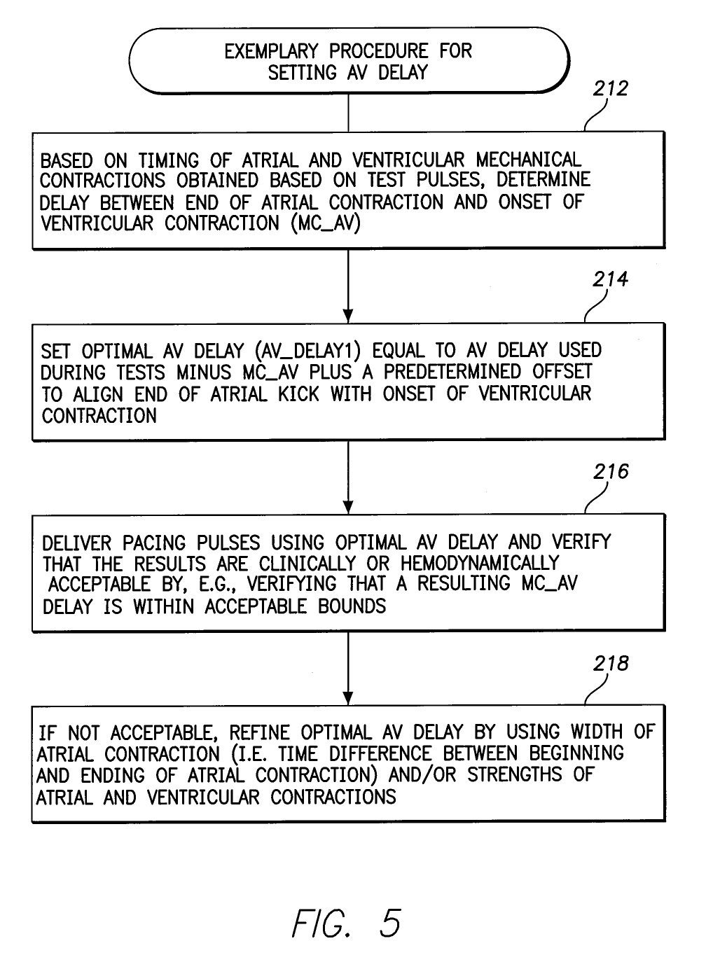

FIG. 2 broadly summarizes the general technique for optimizing AV pacing delays exploited by the components of FIG. 1. Beginning at step 100, the end of atrial mechanical contraction within the heart of a patient is detected based atrial vector impedance values, LAP or other cardiomechanical signal parameters sensed by the device. Exemplary techniques for detecting the end of atrial contraction will be described in detail below with reference to FIG. 3. At step 102, the onset of isovolumic ventricular mechanical contraction is detected within the patient based on impedance, S1 heart sounds, LVP, RVP, PPG signals and/or LAP signals or other suitable cardiomechanical signal parameters sensed by the device. Exemplary techniques for detecting the onset of isovolumic contraction will be described in detail below with reference to FIG. 4. At step 104, a time delay between the end of atrial mechanical contraction and the onset of isovolumic ventricular mechanical contraction is determined. Herein, this time delay is referred to as MC_AV. At step 106, preferred or optimal AV pacing delays are set based on MC_AV so as to, for example, align the end an atrial kick with the onset of isovolumic ventricular mechanical contraction. It is again noted that the term “AV delay” as it is used herein broadly encompasses both AV/PV delays. Exemplary techniques for setting the AV delay will be described in detail below with reference to FIGS. 5-8. Then, at step 108, cardiac pacing is delivered and/or controlled based on the AV pacing delays. This can include CRT.

Thus, FIG. 2 broadly summarizes a cardiomechanical-based AV optimization technique. The optimization can be performed under the control of a clinician operating an external programmer, with the clinician reviewing data received from the implanted device and controlling any reprogramming thereof. For example, the external programmer can process impedance data received from the implanted device to determine MC_AV and recommend optimal AV pacing delays, which are then programmed into the implanted device via telemetry under clinician control. In some implementations, the implanted device itself performs the AV optimization and then reprograms its own AV delays accordingly. That is, some or all of the steps of FIG. 2 can be performed by the implantable device itself, if so equipped.

In some examples, the AV optimization procedure is used in conjunction with IEGM-based AV optimization techniques, such as QuickOpt, which provide baseline AV values. To then program VV pacing delays, IEGM-based VV optimization techniques can be employed, as in some of the aforementioned QuickOpt techniques. Alternatively, the techniques of U.S. patent application Ser. No. ______ of Min, entitled “Systems and Methods for Determining Optimal Interventricular Pacing Delays based on Electromechanical Delays” (A09e1120) can be used (which is fully incorporated by reference herein if filed prior hereto or contemporaneously herewith)

Note also that other external devices beside a device programmer can be used to perform or control the AV optimization, such as bedside monitors or the like. In some embodiments, systems or devices such as the HouseCall™ system or the Merlin@home/Merlin.Net systems of St. Jude Medical are used.

### Exemplary Cardiomechanical Delay-Based AV Optimization Examples

FIG. 3 illustrates exemplary impedance-based techniques for identifying the end of atrial mechanical contraction for use with a device having a multi-pole LV lead. Beginning at step 200, the device sets up windows for detecting atrial Z and ventricular Z based on end of P-wave and peak of QRS. For example, the atrial window may be set based on the contraction of the atria (kick): window starts at the end of P-wave and stops at the peak of QRS—40 ms. The ventricular window may be set based on the contraction of ventricles: window starts at the peak of QRS and stops at the end of the T-wave.

At step 201, the device selects one or more vectors for detecting atrial cardiogenic impedance such as: (1) an electrical current injection vector between RA tip and LV proximal ring and an impedance-responsive voltage sensing vector between RA tip electrode and LV middle ring electrode; (2) a bipolar electrical current injection vector between RA tip and ring electrodes and a bipolar impedance-responsive voltage sensing vector between adjacent LV ring electrodes; or (3) a unipolar electrical current injection vector between an RA electrode and a device housing electrode and a unipolar impedance-responsive voltage sensing vector between a proximal LV ring electrode and the device housing electrode. Hybrid vectors may also be employed. For example, a hybrid Z configuration can be selected such as a large field vector to injected current vector [e.g. (SVC-CAN) or (RV RING to CAN) or (RV RING to SVC COIL)] along with a sensed local cardiogenic impedance (CI) vector [e.g. LV proximal RING to SVC or CAN] for atrial Z signals and multiple LV RINGS to CAN or SVC for ventricular Z signals. See, FIG. 9, for an exemplary multi-pole LV lead wherein these vectors can be selected. Note that a particularly effective tri-phasic impedance detection pulse for use in detecting cardiogenic impedance is described in U.S. patent application Ser. No. 11/558,194 of Panescu et al., entitled “Closed-Loop Adaptive Adjustment of Pacing Therapy based on Cardiogenic Impedance Signals Detected by an Implantable Medical Device.” However, other impedance detection pulses or waveforms may instead be exploited.

Note also that, rather than detecting impedance, other related electrical signals or parameters can instead be exploited, such as admittance, conductance, immittance or their equivalents. This depends, in part, on how these parameters are defined. Impedance is the numerical reciprocal of admittance. Conductance is the numerical reciprocal of resistance. In general, impedance and admittance are vector quantities, which may be represented by complex numbers (having real and imaginary components.) The real component of impedance is resistance. The real component of admittance is conductance. When exploiting the real components of these values, conductance can be regarded as the reciprocal of impedance. Likewise, when exploiting the real components, admittance can be regarded as the reciprocal of resistance. Immittance represents either impedance or admittance. Generally, herein, “impedance signals” broadly encompasses impedance and/or any of these electrical equivalents and those skilled in the art can readily covert one such parameter to another.

At step 202, the device sets baseline or default values for AV and VV. In one example, baseline values of AV=120 milliseconds (ms) and VV=0 ms are used. Alternatively, the device can use IEGM-based optimized AV delays as the baseline values, if available. For example, if the aforementioned QuickOpt techniques have already been used to suggest AV delays, then those AV delays may be used as the baseline values at step 202. QuickOpt techniques are described in several of the patent documents cited above, such as in U.S. Pat. No. 7,248,925, which is incorporated by reference herein in its entirety. Briefly, in one QuickOpt example, both an intrinsic inter-atrial AA conduction delay and an intrinsic AV conduction delay are determined for the patient. The preferred AV delay for use with the patient is determined based on the intrinsic AA conduction delay and the intrinsic AV conduction delay.

At step 202, the device delivers test atrial pacing pulses (A-pulses) to the patient and attempts to detect the resulting atrial contractions within the atrial cardiogenic impedance signals (within the aforementioned atrial windows.) If no atrial contractions detected within the atrial impedance signals, the device incrementally varies AV by 20-40 ms until atrial contractions are detected. If, at step 204, no atrial contractions are detected within a full range of AV delays (such as within a range of 30 ms to 350 ms), the device instead uses non-impedance-based AV optimization techniques to set the AV delays (such as by using LAP.) Assuming, though, that atrial contractions are found within the atrial impedance signals, the device detects the onset, ending and strength of the atrial mechanical contractions within each cardiac cycle. These values may be averaged over a set of cardiac cycles. In one particular example, the onset of the atrial contraction is detected based on the atrial impedance signal rising above an atrial contraction onset detection threshold set relative to a baseline atrial impedance level. Likewise, the end of the atrial contraction can be detected based on the atrial impedance signal falling below an atrial contraction end detection threshold, also set relative to a baseline atrial impedance level. The strength of the atrial contraction might be detected based on the magnitude of the atrial impedance signal (with larger magnitudes being representing greater contraction strength) or based on the value of its peak atrial dZ/dt value (with larger peak dZ/dt values representing greater contraction strength), depending upon clinical confirmation of these proxy correlations. Max derivatives of dP/dt might also be useful as proxies.

FIG. 4 illustrates exemplary impedance-based techniques for identifying the onset of isovolumic ventricular contraction. (For the purposes of FIG. 4, it is assumed that the device has already set the ventricular Z windows as shown in step 200 of FIG. 3.) At step 206, the device selects one more vectors for detecting ventricular cardiogenic impedance such as: (1) bipolar electrical current injection vector between RV tip and LV tip electrodes and a bipolar impedance-responsive voltage sensing vector between LV ring and RV ring electrodes or (2) a bipolar electrical current injection vector between RV tip and ring electrodes and a bipolar impedance-responsive voltage sensing vector between LV tip and ring electrodes.

At step 208, the device again sets baseline or default values for AV and VV. As in the example of FIG. 3, the exemplary values of AV=120 ms and VV=0 ms can be used. Alternatively, as with atrial event detection, the device can use IEGM-based optimized AV delays, such as QuickOpt-suggested AV delays as the baseline values, if available. The device then delivers test ventricular pacing pulses and detects ventricular contractions within the ventricular impedance signals, using the aforementioned ventricular windows. (Unlike the atrial impedance signal processing discussed above, there is typically no concern regarding failure to detect ventricular mechanical contractions via impedance. Hence, the sort of adjustments to AV pacing delays made in step 202 of FIG. 3 are not typically needed for the ventricular processing.) Also during step 208, the device delivers test ventricular pacing pulses (V-pulses) and detects the resulting ventricular contractions within the ventricular impedance signal.

At step 210, the onset, ending and strength of the ventricular mechanical contractions are detected within each cardiac cycle. These values may be averaged over a set of cardiac cycles. In one particular example, the onset of the ventricular contraction is detected based on the ventricular impedance signal rising above a ventricular contraction onset detection threshold set relative to a baseline ventricular impedance level. Likewise, the end of the ventricular contraction can be detected based on the ventricular impedance signal falling below a ventricular contraction end detection threshold, also set relative to a baseline ventricular impedance level. This determination may be made in conjunction with IEGM parameters such as QRS complexes or T-waves. For example, Z in the T-wave to P-wave (T_P) interval can be used as a reference point. Otherwise routine techniques can again be employed to set the values for these thresholds. The strength of the ventricular contraction can be detected based on the magnitude of the ventricular impedance signal (with larger magnitudes representing greater contraction strength) or based on the value of its peak ventricular dZ/dt value. During step 210, the device also detects the electromechanical delay (V_EM) between each V-pulse (delivered during step 208) and the onset of the resulting ventricular contraction. In this regard, V_EM can be used to time the V contraction using pacing pulses since the system already knows AV or PV. Moreover, V_EM can be used to monitor HF progressions independently. V_EM can also be used as additional correction term in QuickOpt™ as disclosed in prior applications.

Note that various other techniques for detecting the onset of ventricular isovolumic mechanical contraction are discussed in the aforementioned co-pending patent application.

FIG. 5 illustrates exemplary techniques for setting the AV delay, which uses the atrial and ventricular contraction parameters detected based on impedance (or detected using any other suitable cardiomechanical detection technique.) At step 212, based on the timing of atrial and ventricular mechanical contractions obtained using the aforementioned test or baseline values, the device determines the delay between end of atrial contraction and the onset of ventricular contraction (MC_AV). Individual values for MC_AV obtained during different cardiac cycles may be averaged over a set of cardiac cycles. At step 214, the device sets a new optimal AV delay (AV_delay1) equal to the AV delay (used during the tests of FIGS. 3 and 4) minus MC_AV plus a predetermined offset (which in some cases is set to zero) to align the end of the atrial kick with the onset of isovolumic ventricular contraction. A suitable value for the offset may be programmed in advance by the clinician or determined based on otherwise routine studies of hemodynamic efficacy. For example, hemodynamic studies can be conducted to identify preferred or optimal offset values for use with various patients. As noted, in some cases, the offset is simply set to zero. Alternatively, the value of the offset could be set based on clinical studies of intra-thoracic and intracardiac impedance signals for the specific patient or populations of patients.

At step 216, the device delivers pacing pulses using the new optimal AV delay and verifies that the results are clinically or hemodynamically acceptable by, e.g., verifying that a resulting MC_AV delay is within acceptable bounds. At step 218, even the MC_AV delay observed while using the new optimal AV delay is found to be acceptable, the device can further refines the optimal AV delay by using the width of the atrial mechanical contraction (i.e. the time difference between onset and ending of atrial contraction), and/or the strengths of the ventricular mechanical contractions. For example, the AV delay may be adjusted in an attempt to shorten the width of either the atrial or the ventricular contraction (or both) and/or to increase the strengths thereof. This can be achieved by varying the AV delay near AV_delay1 while measuring the widths and strengths of the mechanical contractions.

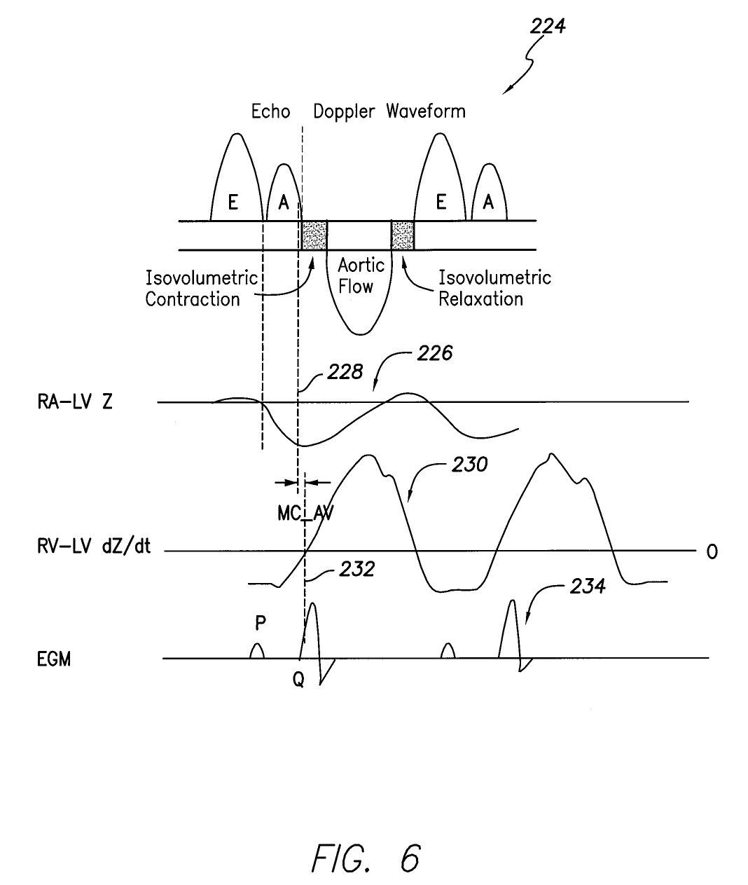

FIG. 6 illustrates an exemplary MC_AV delay along with various other parameters and signals. More specifically, the figure illustrates an echo Doppler waveform 224 for a patient, particularly illustrating intervals of isovolumic ventricular mechanical contraction and isovolumic ventricular mechanical relaxation, as well as an aortic flow interval therebetween. The end of atrial contraction occurs at the end of an interval “A.” This point is identified by vertical line 228. An RA-LV atrial vector impedance (Z) signal 230 is also shown (as might be selected in step 200 of FIG. 3.) The end of atrial contraction occurs at time 228, which corresponds to a trough or valley in the atrial vector impedance signal (i.e. a negative “peak”.) The figure also shows the time rate of change (dZ/dt) of an RV-LV ventricular vector impedance Z signal 230 (as might be selected in step 206 of FIG. 4.) The onset of isovolumic ventricular contraction occurs at time 232, which corresponds to a peak in the rate of change in dZ/dt (i.e. MAX d²Z/dt²).) The interval between time points 228 and 232 is the aforementioned MC_AV delay value detected at step 212 of FIG. 5. Also shown within FIG. 6 is a stylized graph of a surface electrogram (EGM) 234. An IEGM detected by the implantable device has similar timing, though the shape might be different.

### Alternative Cardiomechanical Delay-Based AV Optimization Example

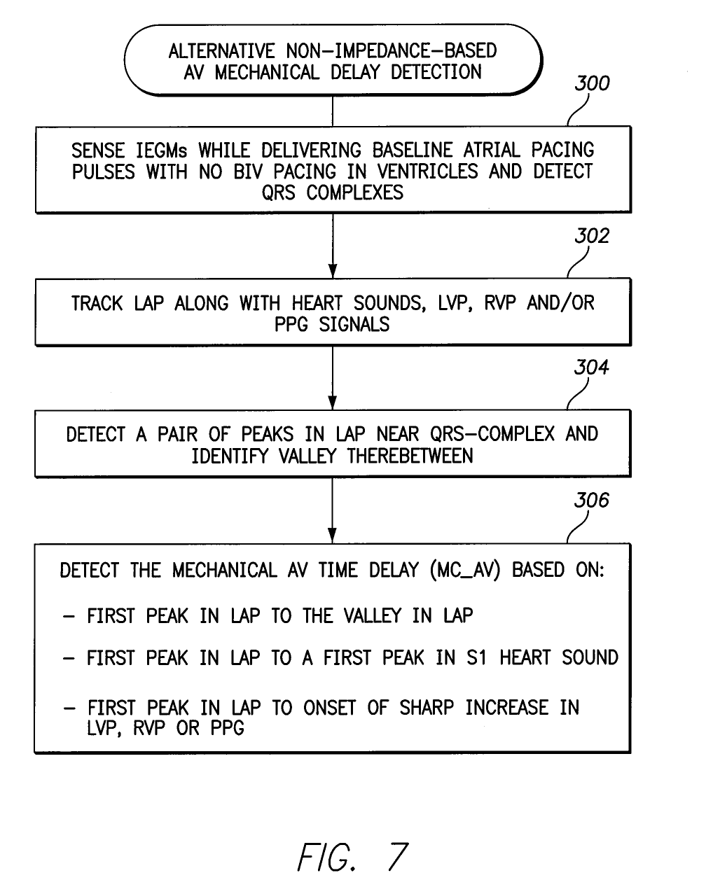

FIG. 7 illustrates an alternative technique for determining MC_AV wherein LAP is exploited along with, in some examples, heart sounds or LVP/RVP and/or PPG signals. Beginning at step 300, the device senses IEGMs while delivering baseline atrial pacing pulses with no biventricular pacing (i.e. VV=0) in the ventricles and while detecting QRS complexes in the LV. At step 302, the device tracks LAP along with, in some examples, heart sounds, LVP, RVP and/or PPG signals. LAP sensors are discussed in, for example, U.S. Published Patent Application 2003/0055345 of Eigler et al., entitled “Permanently Implantable System and Method for Detecting, Diagnosing and Treating Congestive Heart Failure.” Techniques for detecting LAP that do not necessarily require an LAP sensor are discussed in U.S. Provisional Patent Application No. 60/787,884 of Wong et al., entitled, “Tissue Characterization Using Intracardiac Impedances with an Implantable Lead System,” filed Mar. 31, 2006 and U.S. patent application Ser. No. 11/558,101 of Panescu et al., entitled “Systems and Methods to Monitor and Treat Heart Failure Conditions.”

Techniques for detecting heart sounds are discussed, e.g., in U.S. Pat. No. 7,139,609 to Min, et al., entitled “System and Method for Monitoring Cardiac Function via Cardiac Sounds using an Implantable Cardiac Stimulation Device.” See, also, U.S. Pat. No. 6,477,406 to Turcott, entitled “Extravascular Hemodynamic Acoustic Sensor.” Pressure and PPG sensors are discussed, e.g., in U.S. patent application Ser. No. 11/927,026, filed Oct. 29, 2007, entitled “Systems and Methods for exploiting Venous Blood Oxygen Saturation in combination with Hematocrit or other Sensor Parameters for use with an Implantable Medical Device.” See, also, U.S. Pat. No. 6,731,967 to Turcott, entitled “Methods and Devices for Vascular Plethysmography via Modulation of Source Intensity.”

At step 304, the device detects a pair of peaks in LAP near the QRS-complex and identifies a trough or valley therebetween. At step 306, the device then detects or measures the mechanical AV time delay (MC_AV) based on: (1) the first peak in LAP to the valley in LAP; (2) the first peak in LAP to max peak in the S1 heart sound (if sensed) and/or (3) the first peak in LAP to the onset of sharp increase in LVP, RVP or PPG. The various values shown within FIG. 7 are preferably detected for each cardiac cycle. Individual values for MC_AV may be averaged over a set of cardiac cycles.

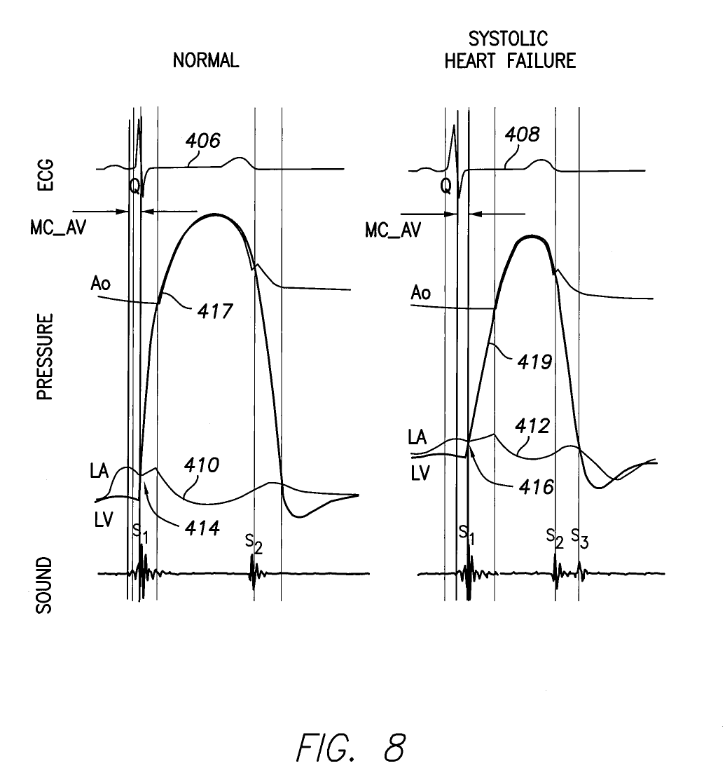

FIG. 8 illustrates electrical cardiac signals for a single cardiac cycle for normal and heart failure patients, 406 and 408, as well as corresponding LAP signals, 410 and 412. LAP signals have two peaks near the QRS or S1 sound. The second peak is aligned with opening of Ao valve and the valley between the two peaks (which is the “first valley” following the QRS) is associated with the onset of isovolumic contraction of both normal and heart failure patients. The first valley within the normal LAP is denoted 414; the first valley within the heart failure LAP is denoted 416. The time delay MC_AV is illustrated, which extends from the first peak in LAP to the middle or the subsequent trough or valley. Note that the figure also shows heart sounds and LVP for the normal patient, 417, and the heart failure patient, 419. At can be seen, the onset of isovolumic ventricular contraction also closely corresponds to the peak in S1 as well as to a point of sharp increase in LVP. As such, these parameters can also be used in combination with LAP (or impedance measurements) to assess MC_AV. If multiple techniques for detecting MC_AV are employed, the values can be averaged to yield a single value for MC_AV for use in optimization.

Thus, FIGS. 2-8 illustrate various exemplary techniques for optimizing AV pacing delays that exploit impedance, heart sounds, LAP, LVP, RVP, PPG signals or other appropriate cardiomechanical signals. Based on the teachings and guidance provided herein, those skilled in the art can identify particular features of these or other cardiomechanical signals that serve to detect the onset or end of atrial and ventricular mechanical contraction.

Note also, that following optimization of the AV delays, the VV delays for the patient may be optimized using techniques described in the aforementioned co-pending application of Min or using other appropriate optimization techniques. It should be understood that the optimal delays obtained using the techniques described herein are not necessarily absolutely optimal in a given quantifiable or mathematical sense. What constitutes “optimal” depends on the criteria used for judging the resulting performance, which can be subjective in the minds of some clinicians. The pacing delays determined by the techniques described herein represent, at least, “preferred” delays. Clinicians may choose to adjust or alter the selection of the delays for particular patients, at their discretion.

Depending upon the particular implementation, some or all of the steps of the various figures are performed by the implantable device itself. Additionally or alternatively, at least some of the steps can be performed by an external programmer or other external system.

Some of the possible advantages of the cardiomechanical-based optimization techniques of FIGS. 2-8 (as compared to some predecessor optimization techniques) are: (1) avoiding the need to use the end of the P-wave of the IEGM for setting AV delays; (2) avoiding the need to determine pacing latency at ventricular leads to correct inappropriate long AV delays for reducing the incidence of AV>AR, since the mechanical contraction of ventricles is now measured directly based on the cardiomechanical parameters; and (3) it is believed that the cardiomechanical-based optimization techniques described herein serve to optimize diastolic filling patterns with fewer assumptions.

Although primarily described with respect to examples having a pacer/ICD equipped to deliver CRT, other implantable medical devices may be equipped to exploit the techniques described. For the sake of completeness, an exemplary pacer/ICD/CRT device will now be described, which includes components for performing the functions and steps already described.

### Exemplary Pacer/ICD/CRT

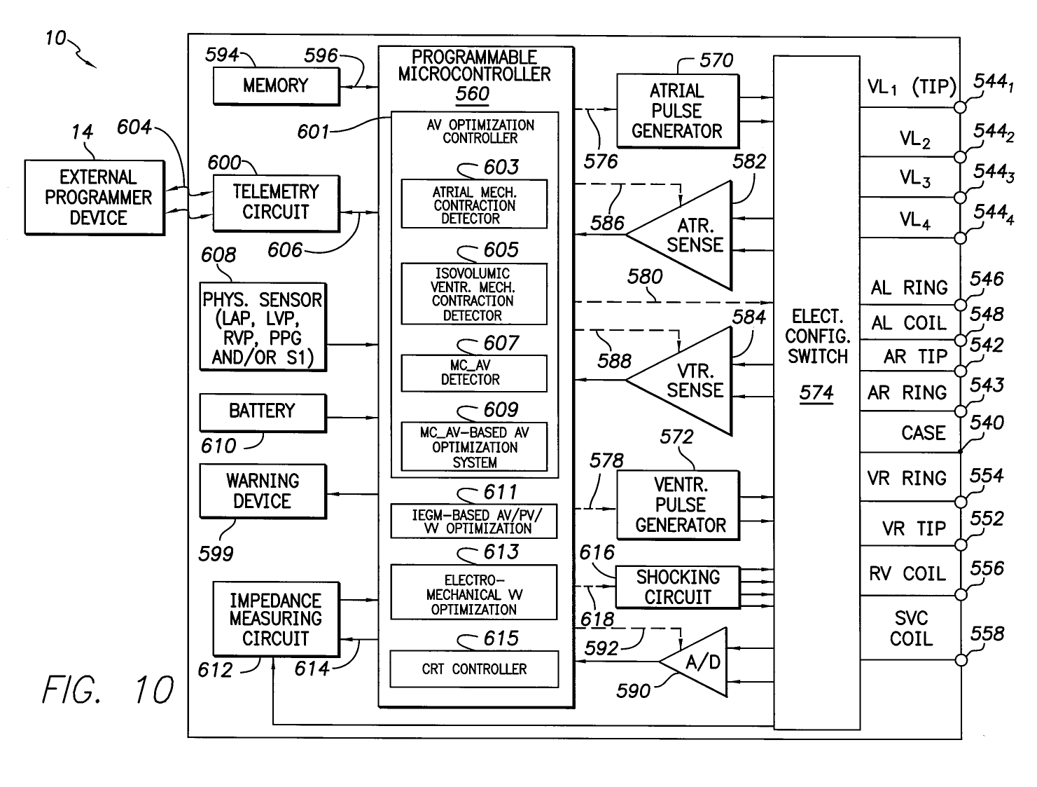

With reference to FIGS. 9 and 10, a description of an exemplary pacer/ICD/CRT will now be provided. FIG. 9 provides a simplified block diagram of the device, which is a dual-chamber stimulation device capable of treating both fast and slow arrhythmias with stimulation therapy, including cardioversion, defibrillation, and pacing stimulation, and also capable of setting and using VV pacing delays, as discussed above, and delivering CRT using the VV delays. To provide other atrial chamber pacing stimulation and sensing, device 10 is shown in electrical communication with a heart 512 by way of a left atrial lead 520 having an atrial tip electrode 522 and an atrial ring electrode 523 implanted in the atrial appendage. Device 10 is also in electrical communication with the heart by way of a right ventricular lead 530 having, in this embodiment, a ventricular tip electrode 532, a right ventricular ring electrode 534, a right ventricular (RV) coil electrode 536, and a superior vena cava (SVC) coil electrode 538. Typically, the right ventricular lead 530 is transvenously inserted into the heart so as to place the RV coil electrode 536 in the right ventricular apex, and the SVC coil electrode 538 in the superior vena cava. Accordingly, the right ventricular lead is capable of receiving cardiac signals, and delivering stimulation in the form of pacing and shock therapy to the right ventricle.

To sense left atrial and ventricular cardiac signals and to provide left chamber pacing therapy, device 10 is coupled to a multi-pole LV lead 524 designed for placement in the “CS region” via the CS os for positioning a distal electrode adjacent to the left ventricle and/or additional electrode(s) adjacent to the left atrium. As used herein, the phrase “CS region” refers to the venous vasculature of the left ventricle, including any portion of the CS, great cardiac vein, left marginal vein, left posterior ventricular vein, middle cardiac vein, and/or small cardiac vein or any other cardiac vein accessible by the CS. Accordingly, an exemplary LV lead 524 is designed to receive atrial and ventricular cardiac signals and to deliver left ventricular pacing therapy using a set of four left ventricular electrodes 526₁, 526₂, 526₃, and 526₄ (thereby providing a quadra-pole lead), left atrial pacing therapy using at least a left atrial ring electrode 527, and shocking therapy using at least a left atrial coil electrode 528. The 526₁ LV electrode may also be referred to as a “tip” or “distal” LV electrode. The 526₄ LV electrode may also be referred to as a “proximal” LV electrode. In other examples, more or fewer LV electrodes are provided. Although only three leads are shown in FIG. 9, it should also be understood that additional leads (with one or more pacing, sensing and/or shocking electrodes) might be used and/or additional electrodes might be provided on the leads already shown, such as additional electrodes on the RV lead.

A simplified block diagram of internal components of device 10 is shown in FIG. 7. While a particular device is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation. The housing 540 for device 10, shown schematically in FIG. 10, is often referred to as the “can,” “case” or “case electrode” and may be programmably selected to act as the return electrode for all “unipolar” modes. The housing 540 may further be used as a return electrode alone or in combination with one or more of the coil electrodes, 528, 536 and 538, for shocking purposes. The housing 540 further includes a connector (not shown) having a plurality of terminals, 542, 543, 544₁-544₄, 546, 548, 552, 554, 556 and 558 (shown schematically and, for convenience, the names of the electrodes to which they are connected are shown next to the terminals). As such, to achieve right atrial sensing and pacing, the connector includes at least a right atrial tip terminal (A_{R} TIP) 542 adapted for connection to the atrial tip electrode 522 and a right atrial ring (A_{R} RING) electrode 543 adapted for connection to right atrial ring electrode 523. To achieve left chamber sensing, pacing and shocking, the connector includes a left ventricular tip terminal (VL₁ TIP) 544₁ and additional LV electrode terminals 544₂-544₄ for the other LV electrodes of the quadra-pole LV lead.

The connector also includes a left atrial ring terminal (A_{L} RING) 546 and a left atrial shocking terminal (A_{L} COIL) 548, which are adapted for connection to the left atrial ring electrode 527 and the left atrial coil electrode 528, respectively. To support right chamber sensing, pacing and shocking, the connector further includes a right ventricular tip terminal (V_{R} TIP) 552, a right ventricular ring terminal (V_{R} RING) 554, a right ventricular shocking terminal (V_{R} COIL) 556, and an SVC shocking terminal (SVC COIL) 558, which are adapted for connection to the right ventricular tip electrode 532, right ventricular ring electrode 534, the V_{R} coil electrode 536, and the SVC coil electrode 538, respectively.

At the core of device 10 is a programmable microcontroller 560, which controls the various modes of stimulation therapy. As is well known in the art, the microcontroller 560 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 560 includes the ability to process or monitor input signals (data) as controlled by a program code stored in a designated block of memory. The details of the design and operation of the microcontroller 560 are not critical to the invention. Rather, any suitable microcontroller 560 may be used that carries out the functions described herein. The use of microprocessor-based control circuits for performing timing and data analysis functions are well known in the art.

As shown in FIG. 10, an atrial pulse generator 570 and a ventricular pulse generator 572 generate pacing stimulation pulses for delivery by the right atrial lead 520, the right ventricular lead 530, and/or the LV lead 524 via an electrode configuration switch 574. It is understood that in order to provide stimulation therapy in each of the four chambers of the heart, the atrial and ventricular pulse generators, 570 and 572, may include dedicated, independent pulse generators, multiplexed pulse generators or shared pulse generators. The pulse generators, 570 and 572, are controlled by the microcontroller 560 via appropriate control signals, 576 and 578, respectively, to trigger or inhibit the stimulation pulses.

The microcontroller 560 further includes timing control circuitry (not separately shown) used to control the timing of such stimulation pulses (e.g., pacing rate, AV delay, atrial interconduction (inter-atrial) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 574 includes a plurality of switches for connecting the desired electrodes to the appropriate I/O circuits, thereby providing complete electrode programmability. Accordingly, the switch 574, in response to a control signal 580 from the microcontroller 560, determines the polarity of the stimulation pulses (e.g., unipolar, bipolar, combipolar, etc.) by selectively closing the appropriate combination of switches (not shown) as is known in the art. The switch also switches among the various LV electrodes.

Atrial sensing circuits 582 and ventricular sensing circuits 584 may also be selectively coupled to the right atrial lead 520, LV lead 524, and the right ventricular lead 530, through the switch 574 for detecting the presence of cardiac activity in each of the four chambers of the heart. Accordingly, the atrial (ATR. SENSE) and ventricular (VTR. SENSE) sensing circuits, 582 and 584, may include dedicated sense amplifiers, multiplexed amplifiers or shared amplifiers. The switch 574 determines the “sensing polarity” of the cardiac signal by selectively closing the appropriate switches, as is also known in the art. In this way, the clinician may program the sensing polarity independent of the stimulation polarity. Each sensing circuit, 582 and 584, preferably employs one or more low power, precision amplifiers with programmable gain and/or automatic gain control, bandpass filtering, and a threshold detection circuit, as known in the art, to selectively sense the cardiac signal of interest. The automatic gain control enables device 10 to deal effectively with the difficult problem of sensing the low amplitude signal characteristics of atrial or ventricular fibrillation. The outputs of the atrial and ventricular sensing circuits, 582 and 584, are connected to the microcontroller 560 which, in turn, are able to trigger or inhibit the atrial and ventricular pulse generators, 570 and 572, respectively, in a demand fashion in response to the absence or presence of cardiac activity in the appropriate chambers of the heart.

For arrhythmia detection, device 10 utilizes the atrial and ventricular sensing circuits, 582 and 584, to sense cardiac signals to determine whether a rhythm is physiologic or pathologic. As used in this section “sensing” is reserved for the noting of an electrical signal, and “detection” is the processing of these sensed signals and noting the presence of an arrhythmia. The timing intervals between sensed events (e.g., AS, VS, and depolarization signals associated with fibrillation which are sometimes referred to as “F-waves” or “Fib-waves”) are then classified by the microcontroller 560 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (A/D) data acquisition system 590. The data acquisition system 590 is configured to acquire intracardiac electrogram signals, convert the raw analog data into a digital signal, and store the digital signals for later processing and/or telemetric transmission to an external device 602. The data acquisition system 590 is coupled to the right atrial lead 520, the LV lead 524, and the right ventricular lead 530 through the switch 574 to sample cardiac signals across any pair of desired electrodes. The microcontroller 560 is further coupled to a memory 594 by a suitable data/address bus 596, wherein the programmable operating parameters used by the microcontroller 560 are stored and modified, as required, in order to customize the operation of device 10 to suit the needs of a particular patient. Such operating parameters define, for example, the amplitude or magnitude, pulse duration, electrode polarity, for both pacing pulses and impedance detection pulses as well as pacing rate, sensitivity, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable device 10 may be non-invasively programmed into the memory 594 through a telemetry circuit 600 in telemetric communication with the external device 602, such as a programmer, transtelephonic transceiver or a diagnostic system analyzer. The telemetry circuit 600 is activated by the microcontroller by a control signal 606. The telemetry circuit 600 advantageously allows intracardiac electrograms and status information relating to the operation of device 10 (as contained in the microcontroller 560 or memory 594) to be sent to the external device 602 through an established communication link 604. Device 10 further includes an accelerometer or other physiologic sensor 608, commonly referred to as a “rate-responsive” sensor because it is typically used to adjust pacing stimulation rate according to the exercise state of the patient. However, the physiological sensor 608 may further be used to detect changes in cardiac output, changes in the physiological condition of the heart, or diurnal changes in activity (e.g., detecting sleep and wake states) and to detect arousal from sleep. Accordingly, the microcontroller 560 responds by adjusting the various pacing parameters (such as rate, AV delay, VV delay, etc.) at which the atrial and ventricular pulse generators, 570 and 572, generate stimulation pulses. While shown as being included within device 10, it is to be understood that the physiologic sensor 608 may also be external to device 10, yet still be implanted within or carried by the patient. A common type of rate responsive sensor is an activity sensor incorporating an accelerometer or a piezoelectric crystal, which is mounted within the housing 540 of device 10. Other types of physiologic sensors are also known, for example, sensors that sense the oxygen content of blood, respiration rate and/or minute ventilation, pH of blood, ventricular gradient, etc. Still further, the sensor may be equipped to detect LAP, LVP, RVP, PPG or S1 heart sounds. It should be understood that multiple separate sensors can be provided and, depending upon the parameter to be detected, at least some of the sensor might be positioned external to the device housing.

The device additionally includes a battery 610, which provides operating power to all of the circuits shown in FIG. 10. The battery 610 may vary depending on the capabilities of device 10. If the system only provides low voltage therapy, a lithium iodine or lithium copper fluoride cell typically may be utilized. For device 10, which employs shocking therapy, the battery 610 should be capable of operating at low current drains for long periods, and then be capable of providing high-current pulses (for capacitor charging) when the patient requires a shock pulse. The battery 610 should also have a predictable discharge characteristic so that elective replacement time can be detected. Accordingly, appropriate batteries are employed.

As further shown in FIG. 10, device 10 is shown as having an impedance measuring circuit 612, which is enabled by the microcontroller 560 via a control signal 614. Uses for an impedance measuring circuit include, but are not limited to, detecting cardiogenic impedance for the purposes of detecting the onset of isovolumic ventricular contraction; lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring respiration; and detecting the opening of heart valves, etc. The impedance measuring circuit 612 is advantageously coupled to the switch 674 so that any desired electrode may be used.

In the case where device 10 is intended to operate as an ICD device, it detects the occurrence of an arrhythmia, and automatically applies an appropriate electrical shock therapy to the heart aimed at terminating the detected arrhythmia. To this end, the microcontroller 560 further controls a shocking circuit 616 by way of a control signal 618. The shocking circuit 616 generates shocking pulses of low (up to 0.5 joules), moderate (0.5-10 joules) or high energy (11 to 40 joules or more), as controlled by the microcontroller 560. Such shocking pulses are applied to the heart of the patient through at least two shocking electrodes, and as shown in this embodiment, selected from the left atrial coil electrode 528, the RV coil electrode 536, and/or the SVC coil electrode 538. The housing 540 may act as an active electrode in combination with the RV electrode 536, or as part of a split electrical vector using the SVC coil electrode 538 or the left atrial coil electrode 528 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 5-40 joules), delivered asynchronously (since R-waves may be too disorganized), and pertaining exclusively to the treatment of fibrillation. Accordingly, the microcontroller 560 is capable of controlling the synchronous or asynchronous delivery of the shocking pulses.

Insofar as the optimization of AV pacing delays is concerned, the microcontroller includes an AV optimization controller 601 operative to perform or control all or some of the AV optimization techniques of FIGS. 2-8 described above. Optimizer 601 includes an atrial mechanical contraction detector 603, an isovolumic ventricular mechanical contraction detector 605 and a MC_AV detector 607, which is operative to detect the time delay from the end of the atrial contraction to the onset of isovolumic ventricular mechanical contraction. These components can additionally detect other features of mechanical contraction, such as contraction strength. An MC_AV-based AV optimization system 609 determines preferred or optimal values for AV based on the techniques discussed above. The optimization techniques can exploit previously optimized AV delay values received via the telemetry circuit or determined by the device itself using an IEGM-based AV/PV/VV optimization system 611, which can exploit the QuickOpt techniques cited above. The microcontroller also includes an electromechanical VV optimization controller 613 operative to perform or control all or some of the VV optimization techniques of the co-pending patent application of Min, incorporated by reference herein. CRT is then controlled by a CRT controller 615. An internal warning device 599 may be provided for generating perceptible warning signals to the patient via vibration, voltage or other methods. Diagnostic data may be recorded in memory 594.

Depending upon the implementation, the various components of the microcontroller may be implemented as separate software modules or the modules may be combined to permit a single module to perform multiple functions. In addition, although shown as being components of the microcontroller, some or all of these components may be implemented separately from the microcontroller, using application specific integrated circuits (ASICs) or the like.

As noted, at least some of the techniques described herein can be performed by (or under the control of) an external device. For the sake of completeness, an exemplary device programmer will now be described, which includes components for controlling at least some of the functions and steps already described.

### Exemplary External Programmer

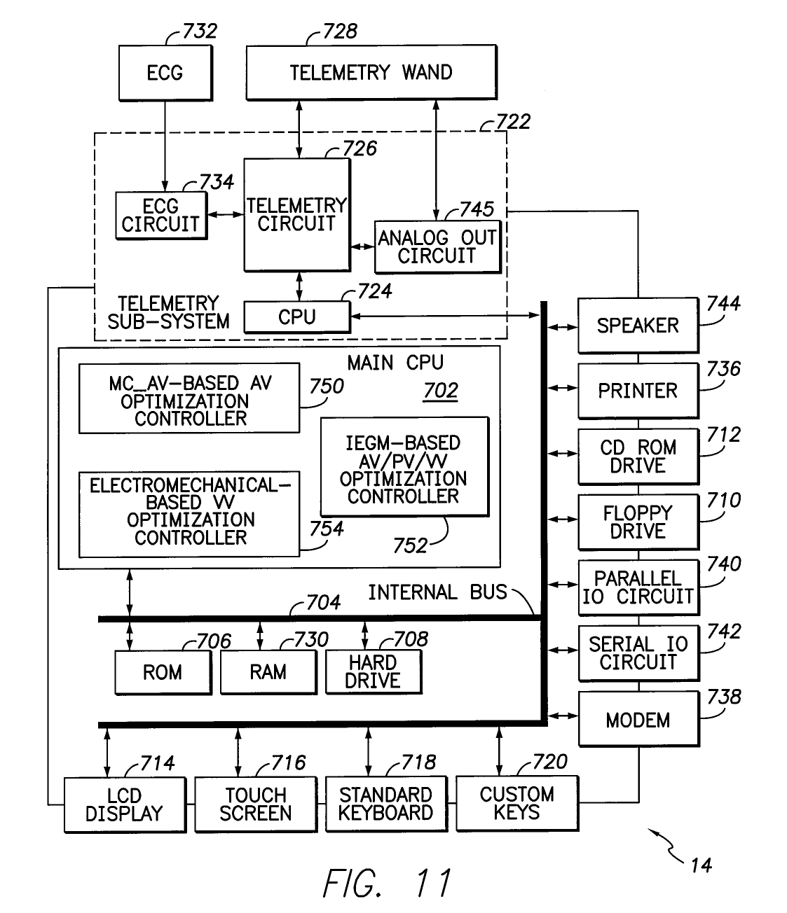

FIG. 11 illustrates pertinent components of an external programmer 14 for use in programming the device of FIG. 10 and for performing or controlling the above-described optimization techniques. For the sake of completeness, other device programming functions are also described herein. Generally, the programmer permits a physician, clinician or other user to program the operation of the implanted device and to retrieve and display information received from the implanted device such as IEGM data and device diagnostic data. Additionally, the external programmer can be optionally equipped to receive and display ECG data from separate external surface ECG leads that may be attached to the patient. Depending upon the specific programming of the external programmer, programmer 14 may also be capable of processing and analyzing data received from the implanted device and from the ECG leads to, for example, render preliminary diagnosis as to medical conditions of the patient or to the operations of the implanted device.

Now, considering the components of programmer 14, operations of the programmer are controlled by a CPU 702, which may be a generally programmable microprocessor or microcontroller or may be a dedicated processing device such as an ASIC or the like. Software instructions to be performed by the CPU are accessed via an internal bus 704 from a read only memory (ROM) 706 and random access memory 730. Additional software may be accessed from a hard drive 708, floppy drive 710, and CD ROM drive 712, or other suitable permanent mass storage device. Depending upon the specific implementation, a basic input output system (BIOS) is retrieved from the ROM by CPU at power up. Based upon instructions provided in the BIOS, the CPU “boots up” the overall system in accordance with well-established computer processing techniques.

Once operating, the CPU displays a menu of programming options to the user via an LCD display 714 or other suitable computer display device. To this end, the CPU may, for example, display a menu of specific programmable parameters of the implanted device to be programmed or may display a menu of types of diagnostic data to be retrieved and displayed. In response thereto, the physician enters various commands via either a touch screen 716 overlaid on the LCD display or through a standard keyboard 718 supplemented by additional custom keys 720, such as an emergency VVI (EVVI) key. The EVVI key sets the implanted device to a safe VVI mode with high pacing outputs. This ensures life sustaining pacing operation in nearly all situations but by no means is it desirable to leave the implantable device in the EVVI mode at all times.

Once all pacing leads are mounted and the pacing device is implanted, the various parameters are programmed. Typically, the physician initially controls the programmer 14 to retrieve data stored within any implanted devices and to also retrieve ECG data from ECG leads, if any, coupled to the patient. To this end, CPU 702 transmits appropriate signals to a telemetry subsystem 722, which provides components for directly interfacing with the implanted devices, and the ECG leads. Telemetry subsystem 722 includes its own separate CPU 724 for coordinating the operations of the telemetry subsystem. Main CPU 702 of programmer communicates with telemetry subsystem CPU 724 via internal bus 704. Telemetry subsystem additionally includes a telemetry circuit 726 connected to telemetry wand 728, which, in turn, receives and transmits signals electromagnetically from a telemetry unit of the implanted device. The telemetry wand is placed over the chest of the patient near the implanted device to permit reliable transmission of data between the telemetry wand and the implanted device. Herein, the telemetry subsystem is shown as also including an ECG circuit 734 for receiving surface ECG signals from a surface ECG system 732. In other implementations, the ECG circuit is not regarded as a portion of the telemetry subsystem but is regarded as a separate component.

Typically, at the beginning of the programming session, the external programming device controls the implanted devices via appropriate signals generated by the telemetry wand to output all previously recorded patient and device diagnostic information. Patient diagnostic information includes, for example, recorded IEGM data and statistical patient data such as the percentage of paced versus sensed heartbeats. Device diagnostic data includes, for example, information representative of the operation of the implanted device such as lead impedances, battery voltages, battery recommended replacement time (RRT) information and the like. Data retrieved from the device also includes the data stored within the recalibration database of the device (assuming the device is equipped to store that data.) Data retrieved from the implanted devices is stored by external programmer 14 either within a random access memory (RAM) 730, hard drive 708 or within a floppy diskette placed within floppy drive 710. Additionally, or in the alternative, data may be permanently or semi-permanently stored within a compact disk (CD) or other digital media disk, if the overall system is configured with a drive for recording data onto digital media disks, such as a write once read many (WORM) drive.

Once all patient and device diagnostic data previously stored within the implanted devices is transferred to programmer 14, the implanted devices may be further controlled to transmit additional data in real time as it is detected by the implanted devices, such as additional IEGM data, lead impedance data, and the like. Additionally, or in the alternative, telemetry subsystem 722 receives ECG signals from ECG leads 732 via an ECG processing circuit 734. As with data retrieved from the implanted device itself, signals received from the ECG leads are stored within one or more of the storage devices of the external programmer. Typically, ECG leads output analog electrical signals representative of the ECG. Accordingly, ECG circuit 734 includes analog to digital conversion circuitry for converting the signals to digital data appropriate for further processing within the programmer. Depending upon the implementation, the ECG circuit may be configured to convert the analog signals into event record data for ease of processing along with the event record data retrieved from the implanted device. Typically, signals received from the ECG leads are received and processed in real time.

Thus, the programmer receives data both from the implanted devices and from optional external ECG leads. Data retrieved from the implanted devices includes parameters representative of the current programming state of the implanted devices. Under the control of the physician, the external programmer displays the current programmable parameters and permits the physician to reprogram the parameters. To this end, the physician enters appropriate commands via any of the aforementioned input devices and, under control of CPU 702, the programming commands are converted to specific programmable parameters for transmission to the implanted devices via telemetry wand 728 to thereby reprogram the implanted devices. Prior to reprogramming specific parameters, the physician may control the external programmer to display any or all of the data retrieved from the implanted devices or from the ECG leads, including displays of ECGs, IEGMs, and statistical patient information. Any or all of the information displayed by programmer may also be printed using a printer 736.

Additionally, CPU 702 also includes an MC_AV-based AV optimization system 750 operative to determine preferred or optimal values for AV pacing based on the cardiomechanical techniques discussed above. As explained, these techniques can exploit an initial or baseline set of AV delay values determined via IEGM-based optimization techniques. Accordingly, an IEGM-based optimization controller 752 may be employed to determine initial values for AV and/or VV delays, which are then used to further refine the AV delays (as well as the VV delays) using the optimization techniques already described. Also, CPU 702 includes an electromechanical VV optimization controller 754 operative to perform or control all or some of the VV optimization techniques of the co-pending patent application of Min, cited above.

Depending upon the implementation, the various components of the CPU may be implemented as separate software modules or the modules may be combined to permit a single module to perform multiple functions. In addition, although shown as being components of the CPU, some or all of these components may be implemented separately using ASICs or the like.

Programmer/monitor 14 also includes a modem 738 to permit direct transmission of data to other programmers via the public switched telephone network (PSTN) or other interconnection line, such as a T1 line or fiber optic cable. Depending upon the implementation, the modem may be connected directly to internal bus 704 may be connected to the internal bus via either a parallel port 740 or a serial port 742. Other peripheral devices may be connected to the external programmer via parallel port 740 or a serial port 742 as well. Although one of each is shown, a plurality of input output (I/O) ports might be provided. A speaker 744 is included for providing audible tones to the user, such as a warning beep in the event improper input is provided by the physician. Telemetry subsystem 722 additionally includes an analog output circuit 745 for controlling the transmission of analog output signals, such as IEGM signals output to an ECG machine or chart recorder.

With the programmer configured as shown, a physician or other user operating the external programmer is capable of retrieving, processing and displaying a wide range of information received from the implanted device and to reprogram the implanted device if needed. The descriptions provided herein with respect to FIG. 11 are intended merely to provide an overview of the operation of programmer and are not intended to describe in detail every feature of the hardware and software of the programmer and is not intended to provide an exhaustive list of the functions performed by the programmer.

In general, while the invention has been described with reference to particular embodiments, modifications can be made thereto without departing from the scope of the invention. Note also that the term “including” as used herein is intended to be inclusive, i.e. “including but not limited to.”

What is claimed is:

1. A method for use with an implantable cardiac rhythm management device for implant within a patient, the method comprising:
detecting an atrial mechanical contraction within the patient based on cardiogenic impedance signals sensed by the device;
detecting an onset of isovolumic ventricular mechanical contractions within the patient based on the cardiogenic impedance signals;
determining a time delay from the end of atrial mechanical contraction to the onset of isovolumic ventricular mechanical contraction;
setting atrioventricular (AV) pacing delays based on the time delay; and
controlling pacing based on the AV pacing delays.

2. The method of claim 1 wherein detecting the atrial mechanical contraction includes detecting cardiogenic impedance signals along at least one atrial impedance vector oriented to detect atrial contractions and then identifying the atrial mechanical contraction within the impedance signals.

3. The method of claim 2 wherein the device is equipped with at least a right atrial (RA) lead and a multi-pole left ventricular (LV) lead and wherein the atrial impedance vectors include an electrical current injection vector between an RA tip electrode and an LV proximal ring electrode and an impedance-responsive voltage sensing vector between the RA tip electrode and an LV middle ring electrode.

4. The method of claim 2 wherein the device is equipped with at least a bipolar RA lead and a multi-pole LV lead and wherein the atrial impedance vectors include a bipolar electrical current injection vector between RA tip and ring electrodes and a bipolar impedance-responsive voltage sensing vector between adjacent LV ring electrodes.

5. The method of claim 2 wherein the device is equipped with at least an RA lead and a multi-pole LV lead and wherein the atrial impedance vectors include a unipolar electrical current injection vector between an RA electrode and a device housing electrode and a unipolar impedance-responsive voltage sensing vector between a proximal LV ring electrode and the device housing electrode.

6. The method of claim 1 wherein detecting the onset of ventricular mechanical contraction includes detecting cardiogenic impedance signals along at least one ventricular impedance vector oriented to detect ventricular contractions and then identifying the onset of ventricular mechanical contraction within the impedance signals.

7. The method of claim 6 wherein the device is equipped with at least LV and right ventricular (RV) leads and wherein the ventricular impedance vectors include a bipolar electrical current injection vector between RV tip and LV tip electrodes and a bipolar impedance-responsive voltage sensing vector between LV ring and RV ring electrodes.

8. The method of claim 6 wherein the device is equipped with at least LV and RV leads and wherein the ventricular impedance vectors include a bipolar electrical current injection vector between RV tip and ring electrodes and a bipolar impedance-responsive voltage sensing vector between LV tip and ring electrodes.

9. The method of claim 1 wherein the device is equipped to employ hybrid vectors that include a large field vector to injected current vector and a sensed local cardiogenic impedance (CI) vector.

10. The method of claim 9 wherein the large field vector includes one or more of an SVC-can vector, an RV ring to can vector and an RV ring to SVC coil vector.

11. The method of claim 9 wherein the sensed local CI vector includes one or more of an LV proximal ring to SVC vector and an LV proximal ring to can vector for detecting atrial impedance (Z) signals and multiple LV electrode to can vectors for detecting ventricular Z signals.

12. The method of claim 1 wherein detecting the atrial mechanical contraction is performed to detect an end of the atrial contraction.

13. The method of claim 12 wherein detecting the end of atrial mechanical contractions includes:
detecting values representative of left atrial pressure (LAP) within the cardiomechanical signals sensed by the device;
detecting an increase in LAP prior to a QRS complex detected within an intracardiac electrogram (IEGM) sensed by the device;
detecting a first subsequent peak in LAP; and
identifying the end of atrial mechanical contraction as coinciding with the peak in LAP.

14. The method of claim 13 wherein setting AV pacing delays based on the time delay from the atrial mechanical contraction to the onset of ventricular mechanical contraction includes:
calculating the time delay (MC_AV) as the delay from a first peak in LAP to a first valley in LAP.

15. The method of claim 1 wherein detecting the onset of ventricular mechanical contractions includes:
detecting values representative of one or more of LAP, RVP, LVP, PPG signals and heart sounds within the cardiomechanical signals sensed by the device; and
detecting the onset of isovolumic ventricular mechanical contraction based on the detected values.

16. The method of claim 1 wherein detecting the ventricular mechanical contraction includes setting a ventricular event detection window.

17. The method of claim 1 wherein all of the steps are performed by the implantable medical device.

18. The method of claim 1 wherein at least some of the steps are performed by an external device based on signals received from the implantable medical device.

19. A system for use with an implantable cardiac rhythm management device for implant within a patient, the system comprising:
an atrial mechanical contraction detection system operative to detect atrial mechanical contractions within the heart of the patient based on cardiomechanical signals detected within the patient by the device;
a ventricular mechanical contraction detection system operative to detect the onset of isovolumic ventricular mechanical contractions within the heart of the patient based on the cardiomechanical signals;
a cardiomechanical time delay determination system operative to determine a time delay between the atrial mechanical contraction and the onset of ventricular mechanical contraction;
an atrioventricular (AV) pacing delay determination system operative to set AV pacing delays based on the time delay; and
a pacing controller operative to control pacing based on the AV pacing delays.

20. A system for use with an implantable cardiac rhythm management device for implant within a patient, the system comprising:
means for detecting atrial mechanical contraction within the heart of the patient;
means for detecting an onset of isovolumic ventricular mechanical contraction within the heart of the patient;
means for determining a time delay between the atrial mechanical contraction and the onset of ventricular mechanical contraction; and
means for setting atrioventricular (AV) pacing delays based on a time delay from the atrial mechanical contraction to the onset of ventricular mechanical contraction.
